(19) United States
(12) Patent Application Publication
(10) Pub. No.: US 2012/0148690 A1
(43) Pub. Date: Jun. 14, 2012

(54) SKIN BARRIER FUNCTION RECOVERY ACCELERATOR

(75) Inventors: Mitsuhiro Denda, Yokohama-shi (JP); Moe Tsutsumi, Yokohama-shi (JP); Sumiko Denda, Yokohama-shi (JP)

(73) Assignee: SHISEIDO COMPANY, LTD.

(21) Appl. No.: 12/964,986

(22) Filed: Dec. 10, 2010

(51) Int. Cl. A61K 36/31; A61K 31/26; A61P 17/00; C07C 47/232; A61K 31/11; C07D 239/10; A61K 31/513; C07C 47/22; C07C 233/05; A61K 31/16; C07C 49/437; A61K 31/125; C07C 35/12; A61K 31/045; C07C 69/84; A61K 31/60; C07C 321/18; A61K 31/105; C07C 331/22

(52) U.S. Cl. 424/755; 558/17; 514/514; 568/425; 514/701; 544/316; 514/274; 568/448; 514/703; 564/209; 514/628; 568/374; 514/692; 568/829; 514/729; 560/64; 514/159; 568/22; 514/707

(57) ABSTRACT

A novel skin barrier function recovery acceleration method and accelerator are provided. The present invention provides a skin barrier function recovery accelerator that comprises a TRPA1 agonist capable of activating TRPA1 in epidermal cells.

### TECHNICAL FIELD

The present invention relates to acceleration of recovery of skin barrier function by activating TRPA1, which is a member of the transient receptor potential (TRP) family.

2. Background Art

Loss of moisture from the skin is known to be much more prominent in comparison with healthy skin in symptoms of rough skin observed in various skin diseases such as atopic dermatitis, psoriasis or contact dermatitis. A decrease in components thought to be involved in retention of moisture in the skin and responsible for the function of the skin as a barrier has been considered to be involved in this increase in so-called transepidermal water loss (TEWL).

Although considerable research has been conducted on substances having the effects of improving and preventing rough skin, there has been insufficient research on substances having the effect of improving or recovering the skin's barrier function, and since the relationship between effects that improve the barrier function of the skin and effects that improve or prevent rough skin has yet to be clarified, substances that effective for improving or preventing rough skin are not necessarily effective for improving the barrier function of the skin.

Several temperature-sensitive receptor proteins have been cloned from the peripheral nervous system as members of the transient receptor potential (TRP) family. Members of the TRP family have been reported to function as sensors of temperature and other physical or chemical factors (Dhaka, et al., Annu. Rev. Neurosci., 29, 135-161 (2006): Non-Patent Document 1), and TRPV1, TRPV3 and TRPV4 have been determined to be present in epidermal keratinocytes (Denda, et al., Biochem. Biophys. Res. Commun., 285, 1250-1252 (2001): Non-Patent Document 2). The inventor of the present invention clearly determined that TRPV1 and TRPV4 are intimately related to homeostasis of the epidermal permeability barrier (Denda, et al., J. Invest. Dermatol., 127, 1713-1719 (2007): Non-Patent Document 3). When TRPV1 is activated with heat (about 43° C.) or capsaicin after having destroyed the skin barrier, repair of the skin barrier was delayed. This delay was inhibited by the TRPV1 antagonist, capsazepine. In contrast, barrier repair was accelerated by activation of TRPV4 by heat (36 to 40° C.) or by a special TRPV4 agonist. This effect was inhibited by the common TRP inhibitor, ruthenium red. These results suggest that TRP receptor plays an important role in homeostasis of the epidermis.

### PRIOR ART DOCUMENTS

### Non-Patent Documents

[Non-Patent Document 1] Annu. Rev. Neurosci., 29, 135-161 (2006)
[Non-Patent Document 2] Biochem. Biophys. Res. Commun., 285, 1250-1252 (2001)
[Non-Patent Document 3] J. Invest. Dermatol., 127, 1713-1719 (2007)
[Non-Patent Document 4] J. Invest. Dermatol., 12 Mar. 2009, dio:10.1038/jid.2009.58
[Non-Patent Document 5] Neuron, 41, 849-857 (2004)
[Non-Patent Document 6] Nature, 445, 541-545 (2007)
[Non-Patent Document 7] Mol. Cell. Neurosci., 32, 335-343 (2006)
[Non-Patent Document 8] Curr. Opin. Neurobiol., 17, 490-497 (2007)
[Non-Patent Document 9] Curr. Biol., 15, 929-934 (2005)
[Non-Patent Document 10] J. Invest. Dermatol., 111, 858-863 (1998)
[Non-Patent Document 11] Proc. Natl. Acad. Sci. USA, 104, 13535-13530 (2007)
[Non-Patent Document 12] Br. J. Dermatol., 127, 654-659 (2007)
[Non-Patent Document 13] Semin. Dermatol., 11(2), 176-82 (1992)
[Non-Patent Document 14] J. Invest. Dermatol., 109, 84-90 (1997)

### SUMMARY OF THE INVENTION

### Problems to be Solved by the Invention

TRPA1 has recently been reported to be expressed in human epidermis (Atoyan, et al., J. Invest. Dermatol., 12 Mar. 2009, dio:10.1038/jid.2009.58: Non-Patent Document 4). The inventor of the present invention hypothesized that TRPA1 is also related to homeostasis of the epidermal barrier, and investigated the effects of TRPA1 agonists and antagonists on homeostasis of the epidermal permeability barrier. In addition, an investigation was also made of the effect of short-term exposure to low temperatures on the speed of barrier recovery.

An object of the present invention is to provide a novel method and accelerator for accelerating recovery of skin barrier function by focusing on the presence of TRPA1 in epidermal cells.

### Means for Solving the Problems

As a result, the inventor of the present invention found that recovery of skin barrier function can be accelerated by activating TRPA1 in epidermal cells.

Thus, in a first aspect thereof, the present invention provides a skin barrier function recovery accelerator that comprises a TRPA1 agonist capable of activating TRPA1 in epidermal cells.

Examples of compounds that activate TRPA1 include allyl isothiocyanate, cinnamaldehyde, icilin, super cinnamaldehyde, SC alkyne, acrolein, pentenal, mustard oil, iodoacetoamide, camphor, menthol, methyl salicylate, gingenol and allicin.

In a second aspect thereof, the present invention provides a method for improving the skin that comprises accelerating recovery of skin barrier function by activating TRPA1 in epidermal cells.

Preferably, activation of TRPA1 in epidermal cells is achieved by exposing the skin to a low temperature for a short period of time, such as exposing for several seconds to several minutes at a temperature of 17° C. or lower, and/or achieved by exposing the skin to a TRPA1 agonist. This skin improvement method may be used for cosmetic purposes or medical purposes.

### Effects of the Invention

The present invention provides a novel skin barrier recovery function accelerator and acceleration method.

### BRIEF DESCRIPTION OF THE DRAWINGS

FIG. 1 is a drawing showing the effects on skin barrier function recovery in the case of allowing TRPA1 agonists and antagonists to act on skin in which skin barrier function has been damaged.

FIG. 2 is a drawing showing the effects on skin barrier function recovery in the case of having exposed skin in which skin barrier function has been damaged to a low temperature.

FIG. 3 consists of immunohistological photographs showing behavior of TRPA1 in the case of having exposed skin in which skin barrier function has been damaged to a low temperature.

FIG. 4 consists of electron micrographs showing recovery of skin barrier function in the case of having exposed skin in which skin barrier function has been damaged to a low temperature.

### EMBODIMENTS OF THE INVENTION

TRPA1 is known to be a member of the TRP super family that is localized in a subset of nociceptive neurons. These receptors are known to be activated in response to low temperatures of about 17° C. As has been previously described, TRPA1 has recently been reported to be expressed in human epidermis.

The present invention is based on the finding that activation of TRPA1 is effective for accelerating recovery of skin barrier function.

Activation of TRPA1 can be achieved by exposing epidermal cells expressing TRPA1 for a short period of time, such as 1 or 2 seconds to 10 minutes, preferably 5 seconds to 5 minutes, more preferably 10 seconds to 3 minutes, even more preferably 20 seconds to 2 minutes and most preferably 30 seconds to 1 minute, to a low temperature of, for example, 17° C. or lower, preferably 0 to 17° C., more preferably 10 to 17° C. and even more preferably 15 to 17° C. Moreover, activation of TRPA1 is also achieved by exposing epidermal cells expressing TRPA1 to a TRPA1 agonist.

Numerous TRPA1 agonists are known, several examples of which are listed as follows: allyl isothiocyanate, cinnamaldehyde (refer to Bandell, et al., Neuron, 41, 849-857 (2004): Non-Patent Document 5 regarding the aforementioned agonists), icilin, super cinnamaldehyde, SC alkyne, acrolein, pentenal, mustard oil, iodoacetoamide (refer to Macpharson, et al., Nature, 445, 541-545 (2007): Non-Patent Document 6 regarding the aforementioned agonists), camphor, menthol (refer to Macpherson, et al., Mol. Cell. Neurosci., 32, 335-343 (2006): Non-Patent Document 7 regarding the aforementioned agonists), methyl salicylate, gingenol (refer to Bandell, et al., Curr. Opin. Neurobiol., 17, 490-497 (2007): Non-Patent Document 8 regarding the aforementioned agonists), and allicin (refer to Macpherson, et al., Curr. Biol., 15, 929-934 (2005): Non-Patent Document 9). In the present invention, the TRPA1 agonist is not limited to the aforementioned commonly known agonists. In addition, these agonists may be used alone or in combination.

Activation of TRPA1 can be achieved by both exposing epidermal cells expressing TRPA1 to a low temperature and exposing to a TRPA1 agonist. In this case, exposure to low temperature and exposure to TRPA1 agonist may be carried out simultaneously or sequentially, and there are no particular limitations on the order thereof.

Test of Skin Barrier Function Recovery Acceleration Effects

Although any TRPA1 agonist has the effect of accelerating recovery of skin barrier function, that effect can be confirmed using various methods, and for example, can be measured quantitatively or qualitatively by carrying out tape stripping the skin of a mammal (such as a human, mouse, rat or rabbit) and evaluating the process by which skin barrier function that has been damaged by that tape stripping recovers to its original state by using transepidermal water loss (TEWL) as an indicator. This measurement can be carried out, for example, in the manner described below.

1. Transepidermal water loss (TEWL) in the vicinity of the back of a hairless mouse is measured using a moisture evaporation measurement apparatus. The value at this time is used as the 100% TEWL recovery rate.

2. The skin barrier is destroyed using cellophane tape and the like and using that tape to peel the stratum corneum from a hairless mouse. At this time, this procedure is preferably repeated until the TEWL value becomes about 800 to 900. The value obtained by subtracting the measured value obtained after peeling the stratum corneum from the measured value obtained prior to peeling the stratum corneum indicates a state in which the skin barrier has suffered the deepest damage, namely a recovery rate of 0%.

3. A suitable amount (such as 100 μl) of a suitable concentration (such as 1 mM) of a test sample is placed on a suitable substrate such as a piece of plastic wrap, affixed to the back of a mammal, and then peeled after the passage of a suitable amount of time (such as 5 minutes).

4. TEWL is then measured using the moisture evaporation measurement apparatus after the passage of suitable amounts of time (such as, 0, 2, 4 and 6 hours). Recovery rate is then calculated by subtracting the TEWL value prior to removing the stratum corneum from the measured value at each measurement time in the same manner as when removing the stratum corneum.

In other words, recovery rate can be determined in accordance with the formula indicated below.

$$Recovery rate ( \% ) = \frac{( \begin{matrix}TEWL at each time after \\ removing stratum corneum\end{matrix} ) - ( \begin{matrix}TEWL before removing \\ stratum corneum\end{matrix} )}{( \begin{matrix}TEWL immediately after \\ removing stratum corneum\end{matrix} ) - ( \begin{matrix}TEWL before removing \\ stratum corneum\end{matrix} )} \times 100$$

In the present invention, “acceleration of recovery of skin barrier function” refers to having the effect of accelerating transepidermal water loss (TEWL) recovery rate by virtue of clearly observing a significant difference in the case of comparing a control with values of TEWL at each measurement time based on a value of 0% for the value of TEWL immediately after tape stripping of the skin and a value of 100% for the value prior to tape stripping.

The TRPA1 agonist relating to the present invention is able to significantly inhibit abnormal skin growth induced by drying irritation, and even reduce the thickness of skin, by accelerating recovery of skin barrier function.

The TRPA1 agonist of the present invention can be incorporated in a cosmetics, pharmaceuticals or quasi-drugs such as ointments, creams, milky lotions, lotions, facial packs or bath additives, and can be preferably applied to the skin as an external skin preparation. Although there are no particular limitations on the incorporated amount thereof, the incorporated amount is about 0.001 mM to 1 M, preferably about 0.01 to 100 mM, and more preferably about 0.1 to 10 mM based on the total amount of base.

### EXAMPLES

Methods

Male hairless mice age 7 to 10 weeks (HR-1, Hoshino Laboratory Animals, Inc., Japan) were used in all experiments. Measurement of skin barrier function, barrier destruction and application of test samples were carried out while the animals were anesthetized with Nembutal. This study was approved by ethics committee of the Shiseido Research Center to comply with guidelines of the U.S. National Institutes of Health (NIH).

Permeability barrier function was evaluated on the basis of measurement results for transepidermal water loss (TEWL) obtained using an electrical moisture analyzer (Meeco Inc.) in the manner previously described (Denda, et al., J. Invest. Dermatol., 111, 858-863 (1998): Non-Patent Document 10). In the barrier recovery experiment, treatment was carried out on the animals consisting of repeatedly carrying out tape stripping on skin on both sides of the abdomen until TEWL reached 7 to 10 mg/cm²/hr. The effects of each treatment were investigated by measuring at two locations on the sides of the abdomen using four mice. Next, TEWL values at the same sites were measured at 1, 3, 6 and 24 hours after barrier destruction. In order to avoid the effects of circadian rhythm, the speed of barrier recovery was investigated immediately after destroying the skin barrier between the hours of 7:00 AM and 8:00 AM. The results for barrier recovery were expressed in terms of recovery rate (%). This is because the degree of barrier destruction varies from day to day. Recovery rate was calculated for each mouse using the following formula:

[(TEWL immediately after barrier destruction−TEWL at indicated time)/(TEWL immediately after barrier destruction−TEWL basal value)]×100%

Immunohistochemistry

After fixing a skin sample by placing in PBS containing 4% paraformaldehyde for 10 minutes at room temperature, washing with PBS solution, and blocking by placing in a blocking solution for 30 minutes, the skin sample was incubated with rabbit anti-TRPA1 antibody (100:1, Abcam Plc.) for 1 hour at room temperature. Next, after washing the skin sample three times for 5 minutes each with PBS solution containing 0.05% Tween 20, and coupling with Alexa Fluor Goat Anti-Rabbit 596 antibody (Molecular Probes, Inc.) for 30 minutes, the skin sample was washed three times for 5 minutes each with PBS solution containing 0.05% Tween 20. Finally, the skin sample was placed on Fluoromount Plus (Diagnostic Biosystems Inc.).

Electron Microscopy

Different mice from those used in the barrier dynamics experiment were used to conduct a histological study. The side of the abdomen was treated with a cotton ball immersed in acetone until TEWL reached 7 to 10 mg/cm²/hr. One hour later, the mice were anesthetized and skin samples were harvested. Four mice were used for each treatment, and five sections were harvested from each skin sample. A skin sample of adequate thickness was sliced into sections (<0.5 mm³) followed by fixing in modified Karnofsky fixative overnight and post-fixing in 2% osmium tetroxide or 2% ruthenium tetroxide. Following fixation, all of the samples were dehydrated in a series of ethanol solutions having different concentrations and embedded in an Epon epoxy mixture. The thin sections were stained with lead citrate and uranyl acetate and observed with an electron microscope. The surface area of the stratum corneum/stratum granulosum (SC/SG) lipid domain was quantified from the materials post-fixed with osmium tetroxide. Measurements were carried out such that it was not possible to know the manner of treatment used in this experiment in advance. Parameters were evaluated using NIH imaging software based on micrographs at the same magnification relating to randomly selected sections.

Results

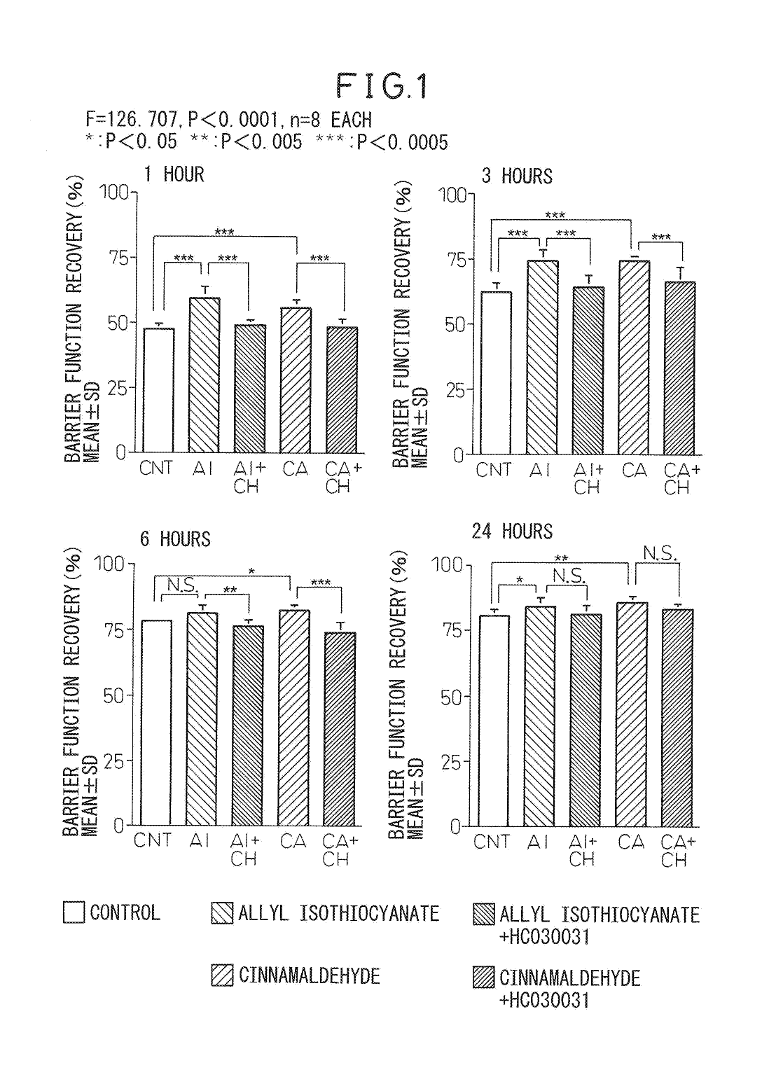

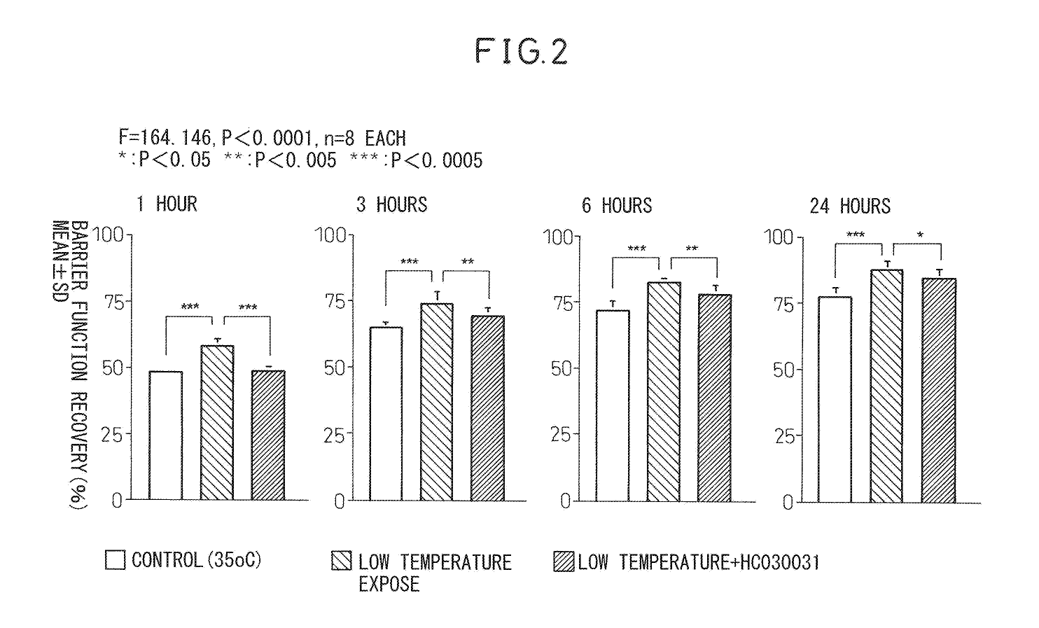

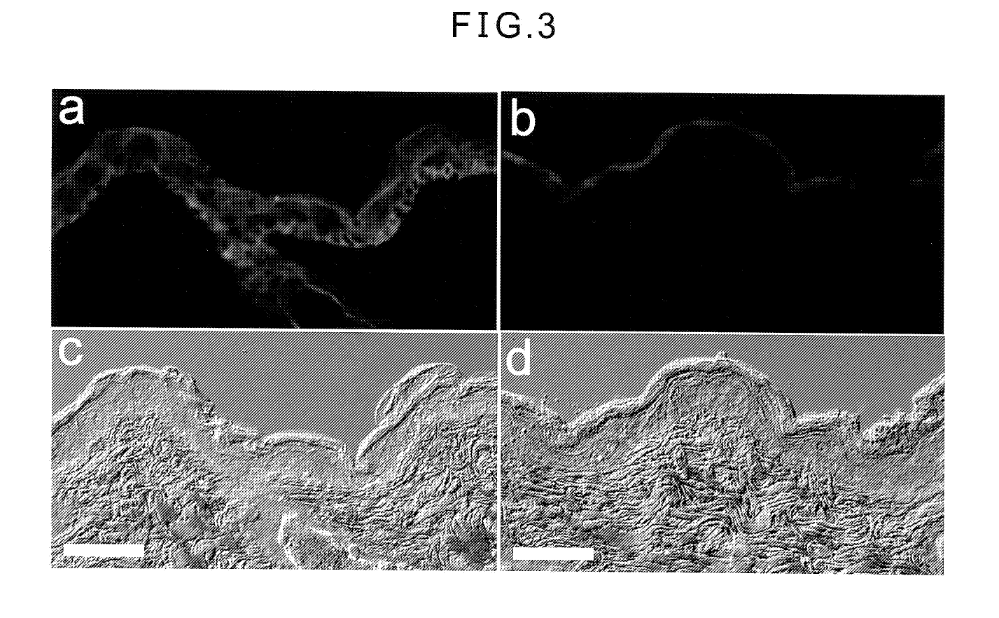

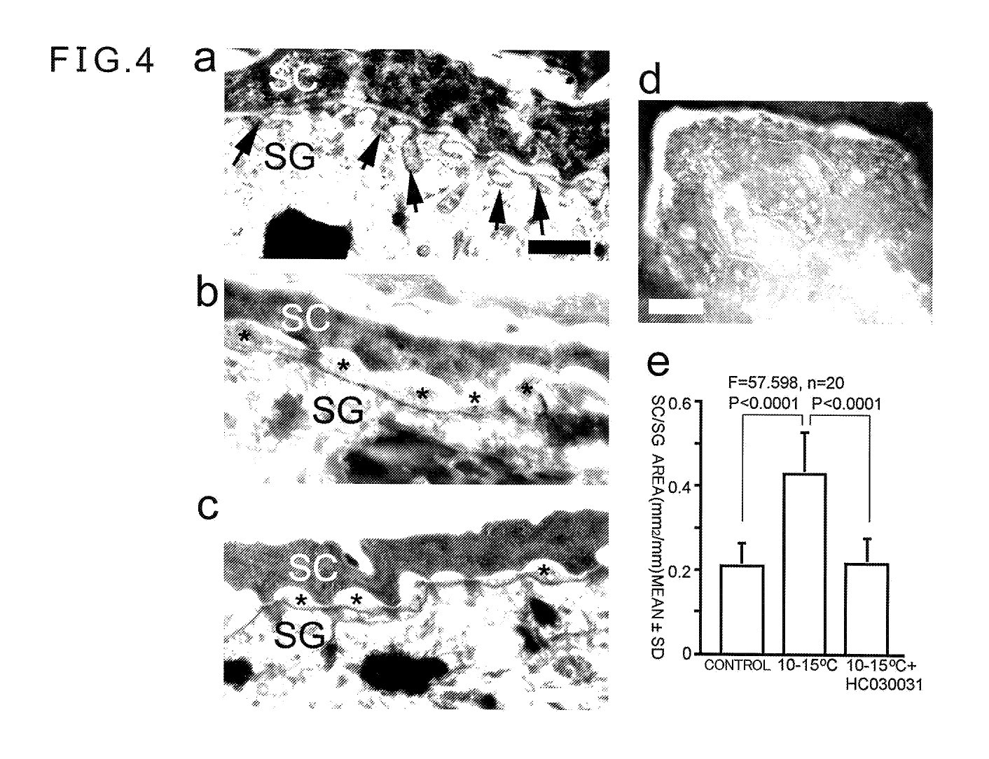

The effects of TRPA1 agonist on undamaged skin of hairless mice were first evaluated. The skin permeability barrier was destroyed by tape stripping followed immediately thereafter by application of 100 μl of agonist solution. Barrier recovery was accelerated by local application of allyl isothiocyanate (0.5 mM) or cinnamaldehyde (0.5 mM), and the effects of both of these reagents were inhibited by pretreatment using the TRPA1 antagonist HC030031: 2-(1,3-dimethyl-2,6-dioxo-1,2,3,6-tetrahydro-7H-purin-7-yl)-N-(4-isopropylphenyl)acetoamide (50 μM) (MacNamara et al., Proc. Natl. Acad. Sci. USA, 104, 13535-13530 (2007): Non-Patent Document 11) (FIG. 1). Next, the surface of the skin on one side of the abdomen was chilled (10 to 15° C.) for 1 minute using a cold insulator. The other side of the abdomen was maintained at 35 to 36° C. using a heating pad. The temperatures of both sides of the abdomen were monitored with a thermometer (Data Logger Thermometer MT-309, Mother Tool Co., Ltd.). Barrier recovery was accelerated after 1 minute of cooling, and that effect was inhibited by pretreatment using HC030031 (FIG. 2). TRPA1 in mouse epidermis was determined to positively immunostained based on the results of the immunohistochemical study (FIGS. 3a and 3c, negative control: FIGS. 3b and 3d). On the basis of the electron microscopy study, although secretion of lamellar bodies between the stratum corneum and stratum granulosum was accelerated by exposure to a low temperature for a short period of time (control of FIGS. 4a and 4b), that acceleration was determined to be inhibited by pretreatment using HC030031 (FIG. 4c). FIG. 4d indicates a lamellar body secreted in skin exposed to a low temperature. A quantified representation of these results is shown in FIG. 4e.

Discussion

Barrier recovery had been reported to be delayed by exposure to low temperatures (Halkier-Sorensen, et al., Br. J. Dermatol., 127, 654-659 (2007): Non-Patent Document 12), and thus the results obtained herein contradict that report. The main difference between these two studies is thought to lie in the duration of exposure to low temperature. Although Halkier-Sorensen, et al. exposed the skin to low temperatures for 3 to 5 hours, in this study, the skin was only exposed for 1 minute. A different study has indicated that lipid formation increases in the epidermis several hours after barrier destruction (Elias and Feingold, Semin. Dermatol., 11(2), 176-82 (1992): Non-Patent Document 13). Long-term exposure to low temperatures has the potential to disturb the various biological processes (such as lipid synthesis) relating to barrier recovery.

Exposure to low temperatures for short periods of time potentially has the possibility of inhibiting various biochemical processes required for barrier recovery. For example, after having increased serine protease activity immediately after having destroyed the skin barrier, barrier recovery was accelerated by an inhibitor of that protease (Denda, et al., J. Invest. Dermatol., 109, 84-90 (1997): Non-Patent Document 14). Thus, exposure to low temperatures for short periods of time has the potential to inhibit various factors that impair barrier homeostasis. However, the effects of TRPA1 agonists and antagonists are suggested to be related to barrier homeostasis of the epidermis.

1. A skin barrier function recovery accelerator, comprising a TRPA1 agonist capable of activating TRPA1 in epidermal cells.

2. The skin barrier function recovery accelerator according to claim 1, wherein the TRPA1 agonist is one or more members selected from the group consisting of allyl isothiocyanate, cinnamaldehyde, icilin, super cinnamaldehyde, SC alkyne, acrolein, pentenal, mustard oil, iodoacetoamide, camphor, menthol, methyl salicylate, gingenol and allicin.

3. A method for improving skin, comprising the step of accelerating recovery of skin barrier function by activating TRPA1 in epidermal cells of a subject in need thereof.

4. The method according to claim 3, wherein activation of TRPA1 in epidermal cells is achieved by exposing the skin of a subject in need thereof to a temperature of 10 to 17° C. for 10 seconds to 5 minutes, and/or achieved by exposing the skin to a TRPA1 agonist.

5. The method according to claim 4, wherein the TRPA1 agonist is one or more members selected from the group consisting of allyl isothiocyanate, cinnamaldehyde, icilin, super cinnamaldehyde, SC alkyne, acrolein, pentenal, mustard oil, iodoacetoamide, camphor, menthol, methyl salicylate, gingenol and allicin.
